(19) United States
(12) Patent Application Publication
(10) Pub. No.: US 2025/0356954 A1
(43) Pub. Date: Nov. 20, 2025

(54) METHODS AND SYSTEMS FOR CONSTRUCTING KNOWLEDGE GRAPHS OF STANDARD DATA ELEMENTS OF BIOMEDICAL DATASETS

(71) Applicant: INSTITUTE OF MEDICAL INFORMATION, CHINESE ACADEMY OF MEDICAL SCIENCES, Beijing (CN)

(72) Inventors: Sizhu WU, Beijing (CN); Zhengyong HU, Beijing (CN); Xiaolei XIU, Beijing (CN); Anran WANG, Beijing (CN)

(73) Assignee: INSTITUTE OF MEDICAL INFORMATION, CHINESE ACADEMY OF MEDICAL SCIENCES, Beijing (CN)

(21) Appl. No.: 19/199,396

(22) Filed: May 6, 2025

(30) Foreign Application Priority Data
May 14, 2024 (CN) 202410595015.0

(51) Int. Cl. G16B 50/10; G06N 5/025; G16B 40/00

(52) U.S. Cl. CPC G16B 50/10; G06N 5/025; G16B 40/00

(57) ABSTRACT

The present disclosure discloses a method and system for constructing a knowledge graph of a standard data element of a biomedical dataset, comprising collecting relevant standard texts of data elements of different types of biomedical datasets and data of a relevant standard of the biomedical dataset; analyzing and summarizing the relevant standard texts of the data elements of the different types of biomedical datasets and the data of the relevant standard of the biomedical dataset; constructing a knowledge model of the knowledge graph of the standard data element of the biomedical dataset; extracting entity type data and attribute data from structured data and an unstructured text in the structured data; and obtaining the knowledge graph of the standard data element of the biomedical dataset by performing knowledge fusion on a plurality of types of data based on an a plurality of types of semantic associative relationships between one or more entity types.

### CROSS REFERENCE TO RELATED APPLICATIONS

The present disclosure claims priority to Chinese Patent Application 202410595015.0, filed on May 14, 2024, the entire contents of which are incorporated herein by reference.

### TECHNICAL FIELD

The present disclosure relates to the field of medical data processing technology, and in particular, to methods and systems for constructing knowledge graphs of standard data elements of biomedical datasets.

### BACKGROUND

Currently, sharing biomedical data has the potential to improve the efficiency of medical research and enhance the transparency of studies. The academic community has also set stringent requirements for research reproducibility and data openness. As a result, more and more biomedical researchers are opting to publicly share their raw data. However, the complexity of biomedical data, particularly in terms of semantics, often leads to challenges such as synonyms and ambiguities. Moreover, the lack of standardized regulations and unified guidelines for data fields and value domains contributes to unclear data semantics, making it difficult to compare datasets or conduct joint analyses across different sources. For example, the English name of the field or variable “gender” in a dataset can be represented as either “gender” or “sex”. In terms of value range, it can be directly represented by text as “male” or “female”, or it can be represented numerically with 0 for male and 1 for female. Without standardized names of data elements and value range specifications, it becomes impossible to integrate or jointly analyze fields or variables with the same semantics across different datasets. Researchers also face difficulties in understanding data semantics, which hampers their ability to effectively utilize the data for analysis, which significantly obstructs data sharing. Therefore, data elements and data element standards of datasets are crucial as they can standardize and unify data structure and semantic expression. However, current data standards are often published in unstructured forms such as PDFs. Many dataset standards in clinical specialties involve 200 to 300 data elements, and different data elements may be defined differently or use different value domains. At present, these standards only provide text-based search, reading, and understanding, making it difficult to effectively utilize them when creating data elements. They are not machine-readable and have poor process ability, which is why these standards are difficult to apply and implement.

Therefore, how to improve machine readability and semantic interoperability while enhancing the usability and utilization of metadata, data elements, classifications, and value domain standards in field-specific datasets is an urgent problem that needs to be addressed by professionals in this field.

### SUMMARY

In view of this, the present disclosure provides a method and system for constructing a knowledge graph of a standard data element of a biomedical dataset, in an aim to collect dataset standards, classifications of data standards, and value domain standards in the field of biomedical science data, performing fragmented and standardized processing, and merging semantic meanings of data elements through part-of-speech and semantic calculations to establish effective associations. Subsequently, a knowledge model of the standard data element of the biomedical dataset is designed and the knowledge graph is constructed to support the standardization of data fields/variables and their value domains. The present disclosure takes the standard data element of the biomedical dataset as an example, and the method and system disclosed can be generalized to the design and implementation of knowledge graphs of data elements of datasets in other fields. On one hand, the method and system disclosed can enhance the field-specific sets of data elements, classification of data elements, and usability and utilization of value domain standards. On the other hand, it is conducive to achieving the unification of data elements and the standardization of establishment of the sets of data elements, refinement, and enrichment of the association between different dataset standards, sets of data elements, data elements, concepts of data elements, and value domains of data elements, thereby improving the machine readability and semantic interoperability.

In order to realize the above purposes, the present disclosure adopts a following technical solution:

One of the embodiments of the present disclosure provides a method for constructing a knowledge graph of a standard data element of a biomedical dataset, comprising:
collecting relevant standard texts of data elements of different types of biomedical datasets and data of a relevant standard of the biomedical dataset;
analyzing and summarizing the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content;
constructing the knowledge model of the knowledge graph of the standard data element of the biomedical dataset, defining one or more entity types, establishing an attribute of each of the one or more entity types and types of semantic associative relationships between the one or more entity types, including:
extracting entity type data and attribute data from structured data and an unstructured text in the structured data;
obtaining the knowledge graph of the standard data element of the biomedical dataset by performing knowledge fusion on a plurality of types of data based on the types of semantic associative relationships between the one or more entity types.

In some embodiments, the structured data and the unstructured text in the structured data are obtained by performing optical character recognition (OCR) on the relevant standard texts of the data elements of the different types of the biomedical datasets and parsing the relevant standard texts using a natural language processing (NLP) manner.

In some embodiments, the method further comprises storing and performing quality inspection on the knowledge graph. The storing includes establishing a plurality of entity attribute tables and a plurality of entity triple relationship tables, performing batch conversion, importing and converting triple data to UTF-8, and storing the knowledge graph using a Neo4j graph database. The quality inspection includes after importing the triple data into the neo4j graph database, performing data sampling to verify correctness of the triple data to ensure correctness of an entity type and a relevance relationship.

In some embodiments, a process for extracting the entity type data and the attribute data from the structured data is as follows:
recognizing and extracting content of the relevant standard texts of the data elements using a human-machine collaboration manner; performing data cleaning, data review, and data quality control on the extracted content, writing a regular expression of identifier data according to a clearly defined coding rule, performing spelling check and quality control on different codes, correcting a problematic identifier, and unifying identifiers; if the extracted content includes a recognition error, useless space or line break, or a garbled code or omission, supplementing and modifying, by human beings, the extracted content to complete extraction and organization of the content of the relevant standard texts and form preliminary structured data.

In some embodiments, a process for extracting the entity type data and the attribute data from the unstructured text in the structured data is as follows:
manually annotating and performing review and quality control on the entity type by recognizing, extracting, and annotating from the unstructured text in the structured data using a subject vocabulary or machine learning manner.

In some embodiments, the types of semantic associative relationships between the one or more entity types include: a relationship between data standards, a relationship between a set of data elements and the data elements, a relationship between the data elements and concepts of the data elements, a relationship between the date elements, a relationship between the data elements and value domains of the data elements, a relationship between a dataset standard and a medical scale/questionnaire, and a relationship between the data elements and the medical scale/questionnaire. The relationship between data standards is pluralistic; the data standards and the dataset of data elements are in an inclusion relationship, and the dataset of data elements and the data elements are in an inclusion relationship, the dataset of data elements includes a plurality of data elements. The relationship between the data elements includes a synonymous relationship, a relevant relationship, and an irrelevant relationship. The value domains of the data elements are classified into four types including an enumeration with external reference type, an enumeration with internal reference type, an enumeration defined within a standard type, and a non-enumerated type based on a source of the value domains and a usage manner. The medical scale is used in the dataset standard, and a scale name and information are extracted from a text, and a connection between a specific medical scale and the data element is established by complementing resources of the medical scale; each of the data elements is a storage name in a standardized dataset of the medical scale, and an association between the data elements and the specific medical scale is established.

In some embodiments, a process for determining the relationship between the data elements includes:
after identifying concepts of the data elements, performing synonymous relationship recognition on the data elements, and if the concepts of two data elements in the data elements are the same in each subject vocabulary of a same medical field, determining the two data elements in a synonymous relationship, and marking a similarity between the two data elements as 1;
if the two data elements are in a non-synonymous relationship, performing similarity calculation on the two data elements with completely different standard codes and data element identifiers using a Jaccard similarity manner by determining a ratio of an intersection set to a concatenation set between sets corresponding to the two data elements, respectively, wherein a calculation formula is as follows:

$$Sim\_ele ( E 1 , E 2 ) = \frac{\vert A ⋂ B \vert}{\vert A \vert + \vert B \vert - \vert A ⋂ B \vert}$$

where, E1 and E2 denote the two data elements, respectively, a tokenization processing is performed on a text of each of the two data elements, E denotes a tokenized text composed of a data element name and a data element definition of each of the two data elements, Sim_ele_name( ) denotes a data element similarity, A denotes a tokenized text of E1, and B denotes a tokenized text of E2, and a final similarity result is controlled to be in a range of [0, 1];
if the two data elements are in the non-synonymous relationship, calculating a similarity between a first data element and a second data element in the two data elements according to the calculation formula; if the similarity between the two data element s is greater than a data element synonymity threshold, determining the first data element and the second data elements being in a candidate synonymity relationship;
if the similarity between the two data elements is greater than a data element relevance threshold and less than the data element synonymity threshold, determining the first data element and the second data element being in a candidate relevance relationship;
if the similarity between the data elements is less than the data element relevance threshold, recording the similarity between the two data elements, and marking a relationship between the first data element and the second data element as irrelevant;

In some embodiments, a process for determining the relationship between the data elements and the value domains of the data elements and determining the types of the value domains includes:
determining, based on the data element and a value domain corresponding to the data element, whether an allowed value of the data element includes a standard number or a number or name of a value domain code table, judging through a coding rule base, and in response to determining the allowed value including the standard number or the number or name of the value domain code table, determining the value domain of the data element as enumeration reference; or in response to determining the allowed value not including the standard number or the number or name of the value domain code table, performing following steps including:
if the value domain of the data element is enumeration reference, further judging if a standard number of a dataset of the value domain is the standard number of the data element or if a number of a value domain code table of the value domain is the number of the value domain code table of the data element, and in response to determining the standard number of the dataset being different from the standard number of the data element or the number of the value domain code table of the value domain not including the number of the value domain code table of the data element, determining the value domain of the data element as the enumeration with external reference type; and in response to determining the standard number of the dataset being the same as the standard number of the data element or the number of the value domain code table of the value domain including the number of the value domain table of the data element, determining the value domain of the data element as the enumeration with internal reference type;
in response to determining the allowed value of the data element not including the standard number or the number or name of the value domain code table and the allowed value includes “;” determining a split numeric item as the enumeration defined within a standard type; and
in response to determining the split numeric item not belonging to the enumeration defined within a standard type, determining the split numeric item as the non-enumerated type.

In some embodiments, performing the knowledge fusion on the plurality of types of data specifically includes:
disambiguating using a pre-existing unique code, including processing a cross-level number;
standardizing a name, including normalization of naming and coding through regulations and standards including WS/T306 Rules for Classification and Coding of Health Information Datasets, WS370-2012 Rules for Formulating Specifications for the Preparation of Basic Health Information Datasets, an institutional specification library and a field vocabulary, similarity calculation, manual verification, and quality control; semantically merging a term and acronym through a field subject vocabulary and a general subject vocabulary;
merging names of the data elements through similarity calculation between the data elements, merging of the concepts of the data elements, and manual discrimination; and
merging names of data value domain tables, wherein a value domain table in a standard text of a dataset and the allowed value of the data element are both related to a relevant name of the data value domain table including a table number, a table code, and a table name, and the merging names of data value domain tables including: performing structured processing on the table number, the table code, and the table name, correcting, combining, and merging the table number, the table code, and the table name, and fusing a standard number to realize merging and disambiguation of the names of the data value domain tables.

One of the embodiments of the present disclosure provides a system for constructing a knowledge graph of a standard data element of a biomedical dataset, comprising following modules:
a data collection module, configured to collect relevant standard texts of data elements of different types of biomedical datasets and data of a relevant standard of the biomedical dataset;
a data analysis module, configured to analyze and summarize the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content;
a knowledge model construction module, configured to construct the knowledge model of the knowledge graph of the standard data element of the biomedical dataset, define one or more entity types, establish an attribute of each of the one or more entity types and types of semantic associative relationships between the one or more entity types;
an entity type extraction module, configured to extract entity type data and attribute data from structured data and an unstructured text in the structured data; and
a knowledge graph obtaining module, configured to obtain the knowledge graph of the standard data element of the biomedical dataset by performing knowledge fusion on a plurality of types of data based on the types of semantic associative relationships between the one or more entity types.

### BRIEF DESCRIPTION OF THE DRAWINGS

In order to more clearly illustrate the technical solutions in the embodiments or prior art of the present disclosure, the accompanying drawings required to be used in the descriptions of the embodiments or prior art will be briefly described hereinafter, and it will be obvious that the accompanying drawings in the following descriptions are only of the present disclosure, and that a person of ordinary skill in the art can obtain other accompanying drawings according to the accompanying drawings provided without exerting creative labor.

FIG. 1 is a flowchart illustrating a method for constructing a knowledge graph of a standard data element of a biomedical dataset according to some embodiments of the present disclosure;

FIG. 2 is a schematic diagram illustrating an exemplary structure of a knowledge model of a knowledge graph of a standard data element of a biomedical dataset according to some embodiments of the present disclosure;

FIG. 3 is a schematic diagram illustrating example data of a portion of a knowledge graph according to some embodiments of the present disclosure; and

FIG. 4 is a schematic diagram illustrating relationships between dataset standards, sets of data elements, data elements, and value domain codes according to some embodiments of the present disclosure.

### DETAILED DESCRIPTION

The technical solutions in the embodiments of the present disclosure will be clearly and completely described below in conjunction with the accompanying drawings in the embodiments of the present disclosure, and it is clear that the embodiments described are only a portion of the embodiments of the present disclosure, and not all of the embodiments. Based on the embodiments in the present disclosure, all other embodiments obtained by a person of ordinary skill in the art without making creative labor fall within the scope of protection of the present disclosure.

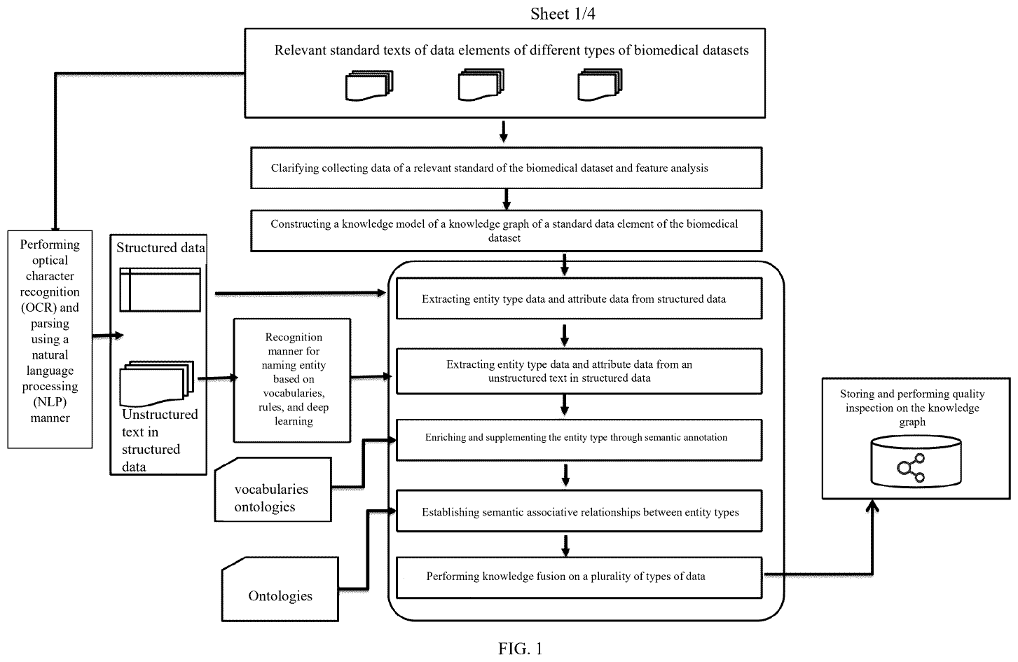

FIG. 1 is a flowchart illustrating a method for constructing a knowledge graph of a standard data element of a biomedical dataset according to some embodiments of the present disclosure. The method may be executed by a processor. The processor may be located on any terminal device. The terminal device may include a computer, a tablet, a cell phone, or the like.

As shown in FIG. 1, embodiments of the present disclosure disclose the method for constructing a knowledge graph of a standard data element of a biomedical dataset (hereinafter referred to as the method), including step 110 to step 130.

Step 110, collecting relevant standard texts of data elements of different types of biomedical datasets and data of a relevant standard of the biomedical dataset.

The biomedical dataset may be a set including various data within a biomedical field. For example, the biomedical dataset may include various forms of data collected in processes of biomedical research, clinical practice, health monitoring, or the like.

Different types of biomedical datasets may be understood as biomedical datasets from different data sources.

In some embodiments, the biomedical dataset may include a plurality of data elements.

The data element may be a storage name in a standardized database of a medical scale, and is configured to establish an association between the data element and a particular medical scale. The medical scale may be used to assist in evaluating the severity of a patient's disease. The data element may standardize and normalize various data in the biomedical dataset, implementing data exchange between different systems more convenient.

The standard text refers to a text that has been normalized or standardized. For example, the standard text may include documents in formats such as.pdf, .doc, and so on.

The data of the relevant standard of the biomedical dataset may be data related to the relevant standard of the biomedical dataset. The relevant standard of the biomedical dataset may include, but not limited to, a dataset standard, a classification standard, a coding standard, and a value domain code standard, as well as relevant external resources involved in a relevant standard of an extended biomedical dataset.

The relevant external resources may include scientific literature, medical glossaries (ICD, UMLS, etc.), etc. A level of the relevant standard of the biomedical dataset may include a national standard, an industry standard, a local standard, and a group standard. The dataset standard may include standards of various types of general datasets, such as a directory of health information data elements, a value domain code of health information data elements, a basic dataset for disease control, a basic information dataset, a basic dataset of medical service, and a basic dataset of electronic medical record (EMR), and may also include standards of specialized disease datasets, such as those for orthopedics, traditional Chinese medicine, hypertension, or the like.

Step 120, analyzing and summarizing the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content.

The standard data element refers to a data element that has undergone standardization. The knowledge graph refers to a data structure that organizes standard data elements in a form of nodes and relationships. The knowledge graph can help users better understand and work with complex standard data elements.

In some embodiments, the processor may analyze and summarize raw data involved in the relevant standard texts regarding provisions in the data of the relevant standards of the biomedical dataset, so as to obtain analyzed and summarized data. For example, if the data of the relevant standard of the biomedical dataset includes Compilation Standard of Basic Health Information Dataset, the processor may, according to the Compilation Standard of Basic Health Information Dataset, analyze and summarize raw data from relevant standard documents of the data elements to obtain analyzed and summarized data.

Parsing the data refers to converting the analyzed and summarized data into data in a form that is easy to understand and process. For example, parsing the data involves cleaning, transforming, or formatting the analyzed and summarized data.

Extracting fine-grained content refers to a process for decomposing the parsed data into a plurality of portions and extracting feature information. The feature information may include, for example, differences in images displayed on different image data. The feature information may be used for fine-grained analysis to reveal a pattern in data.

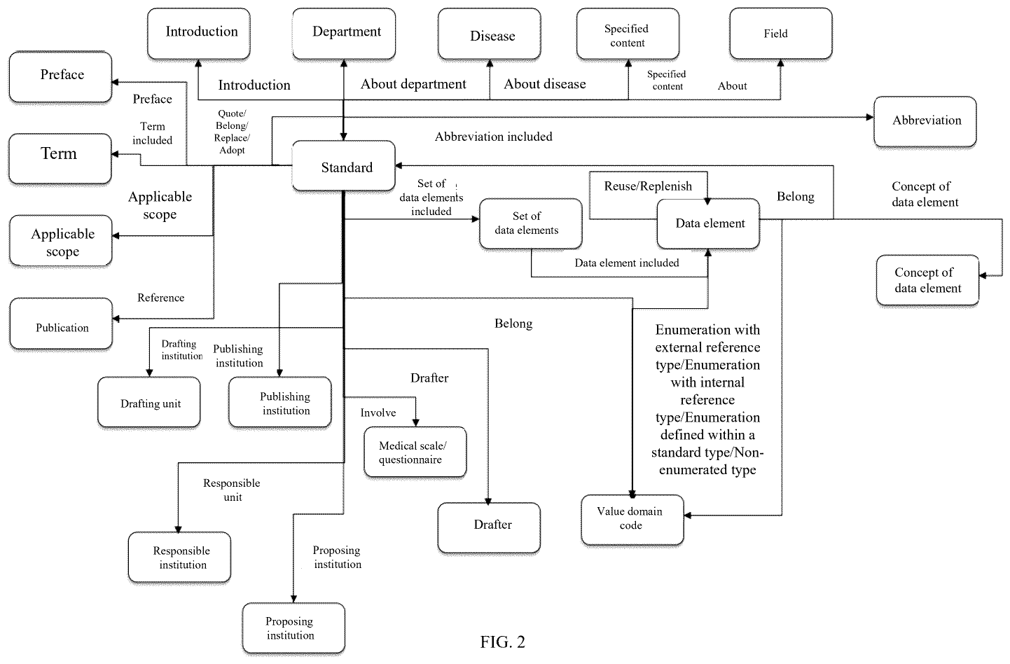

FIG. 2 is a schematic diagram illustrating an exemplary structure of a knowledge model of a knowledge graph of a standard data element of a biomedical dataset according to some embodiments of the present disclosure.

In some embodiments, the knowledge model may be a knowledge model as shown in FIG. 2.

Step 130, constructing the knowledge model of the knowledge graph of the standard data element of the biomedical dataset, defining one or more entity types, establishing an attribute of each of the one or more entity types and types of semantic associative relationships between the one or more entity types.

The entity type in the knowledge model of the knowledge graph of the standard data element of the biomedical dataset may include, but not limited to, 21 types including a standard, terminology, abbreviation, specified content, applicable scope, preface, introduction, sets of data elements, data element, concept of data elements, value domain code, disease, domain, department, publication, responsible institution, proposing institution, drafting institution, etc. At the same time, the attribute of each of the one or more entity types and the types of semantic associative relationships between the one or more entity types may be established. More content about the set of data elements can be referred to the related descriptions later.

The attribute of the entity type refers to a characteristic that the entity type has. For example, the attribute of the entity type includes that a data standard and a set of data elements are in an inclusion relationship, the set of data elements and the data elements are in an inclusion relationship, the set of data elements includes a plurality of data elements, or the like.

In some embodiments, a defined entity type and a defined attribute of each entity type may be predefined for those skilled in the art based on experience.

In some embodiments, the types of semantic associative relationships between the one or more entity types include: a relationship between data standards, a relationship between a set of data elements and the data elements, a relationship between the data elements and concepts of the data elements, a relationship between the date elements, a relationship between the data elements and value domains of the data elements, a relationship between a dataset standard and a medical scale/questionnaire, and a relationship between the data elements and the medical scale/questionnaire.

In some embodiments, more content about a process for establishing the types of semantic associative relationships between the one or more entity types can be found in the description below entitled “a process for establishing types of semantic associative relationships between one or more entity types”.

In some embodiments, a processor may construct the knowledge model of the knowledge graph of the standard data element of the biomedical dataset through step 131 to step 132. Step 131, extracting entity type data and attribute data from structured data and an unstructured text in the structured data.

The structured data refers to a database including a set of data of a specific data type, for example, a medical Hospital Information System (HIS) database. The unstructured text refers to textual content that does not have a fixed format or regularity. The unstructured text includes, for example, mail, news, blogs, emails, and so on, which can exist in text formats such as .pdf and .doc.

The entity type data refers to data related to the entity type, e.g., a preface, an introduction, etc., in the unstructured text. The attribute data may be data related to the attribute of the entity type. For example, since the patient's age has only one value and belongs to a scalar attribute, if the patient's age is recorded in the unstructured text, the patient's age is determined as the attribute data.

In some embodiments, the processor may obtain the structured data and the unstructured text in the structured data by performing optical character recognition (OCR) on relevant standard texts of data elements of different types of biomedical datasets and parsing the relevant standard texts using a natural language processing (NLP) manner.

Optical character recognition (OCR) refers to a technology that scans and recognizes a text on documents and converts the recognized text into a digital text format that may be edited and processed by a computer. The documents may include word documents, pdf documents, or the like.

The natural language processing (NLP) manner enables language interaction between humans and computers, as well as implements text processing, language analysis, text mining, and other tasks.

For more content about how to extract the entity type data and the attribute data from the structured data and the unstructured text in the structured data, please refer to related descriptions of “a specific process for extracting entity type data and attribute data from structured data” and related descriptions of “a specific process for extracting entity type data and attribute data from an unstructured text in structured data”.

Step 132, obtaining the knowledge graph of the standard data element of the biomedical dataset by performing knowledge fusion on a plurality of types of data based on the types of semantic associative relationships between the one or more entity types.

The knowledge fusion refers to a process for integrating information of a same data element in biomedical datasets from different data sources to obtain more comprehensive information about the data element. More content about how to perform the knowledge fusion on the plurality of types of data can be referred to step c1 to step c4 below.

The present disclosure analyzes and summarizes structures of standards at different levels and fields and features of standard data elements of important datasets to support to construct the knowledge model of the knowledge graph of the standard data element of the biomedical dataset, parse the data, and extract the fine-grained content.

Specifically, a core of the construction of the knowledge graph of the standard data element of the biomedical dataset lies in the design and construction of a graph knowledge model oriented to specific needs. Although a small portion of existing studies focus on the construction of knowledge graphs for general standard texts, issues such as coarse knowledge granularity, low standardization, and weak association are common. There is a lack of fine-grained framework modeling, knowledge extraction, and establishment of association specifically tailored to particular fields and applications. Additionally, the construction of machine-readable dataset standards, reuse of data element and value domains remain insufficient. During data processing and construction of graphs, the present disclosure mainly refers to a standard of ISO/IEC 11179 Metadata Registration System, Compilation Standard of Basic Health Information Dataset, and other relevant guidelines and is aimed to satisfy business needs and development goals such as the construction of dataset standards, management, integration, usage, reuse, creation, and comparison of data elements in the biomedical field, and designs the knowledge model of the knowledge graph of the standard data element of the biomedical dataset, thereby realizing fine-grained decomposition and semantic enrichment of biomedical dataset standards. The knowledge model may include, but not limited to, a total of 21 entity types and 30 relationship types, which may be further extended based on specific needs to establish fine-grained associations between different types of standards, content units, and resources, as well as to determine a degree of association between specific entities.

The present disclosure provides a method and a system for constructing a knowledge graph of a standard data element of a biomedical dataset. The method may include collecting relevant standard texts of data elements of different types of biomedical datasets and data of a relevant standard of the biomedical dataset; analyzing and summarizing the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content; constructing the knowledge model of the knowledge graph of the standard data element of the biomedical dataset, defining one or more entity types, establishing an attribute of each of the one or more entity types and types of semantic associative relationships between the one or more entity types, including: extracting entity type data and attribute data from structured data and an unstructured text in the structured data; and obtaining the knowledge graph of the standard data element of the biomedical dataset by performing knowledge fusion on a plurality of types of data based on the types of semantic associative relationships between the one or more entity types The method disclosed in the present disclosure not only enhances the unification of metadata and data elements, utilization of classification of data element and value domain standards, but also helps achieve the unification of data elements, as well as the standardization and refinement of the establishment of the datasets, and enrichment of associations across dataset standards, sets of data elements, data elements, concepts of data elements, and value domains of data elements, thereby improving machine readability and semantic interoperability.

In some embodiments, the method further includes storing and performing quality inspection on the knowledge graph. The storing may include establishing a plurality of entity attribute tables and a plurality of entity triple relationship tables, performing batch conversion, importing and converting triple data to UTF-8, and storing the knowledge graph using a Neo4j graph database. The quality inspection may include after importing all triple data into the Neo4j graph database, performing data sampling to verify correctness of the triple data to ensure correctness of an entity type and a relevance relationship.

The entity attribute table refers to a table used to represent a correspondence between an entity type and a corresponding attribute.

The tripe relationship table may be configured to represent semantic relationships between different entities. The triple relationship table may be a basic building unit of the knowledge graph.

Triple data refers to a data structure including three elements. The three elements may include a subject, a predicate, and an object.

In some embodiments, a person skilled in the art may predefine the plurality of entity attribute tables and the plurality of entity triple relationship tables based on experience.

UTF-8refers to a variable-length character encoding manner for representing all characters in the Unicode character set.

The Neo4j graph database may be understood as a high-performance NoSQL graph database including the triple data.

More content about the related descriptions can be referred to the descriptions below.

In some embodiments, a specific process for extracting the entity type data and the attribute data from the structured data includes:
recognizing and extracting content of the relevant standard texts of the data elements using a human-machine collaboration manner; performing data cleaning, data review, and data quality control on the extracted content, writing a regular expression of identifier data according to a clearly defined coding rule, performing spelling check and quality control on different codes, correcting a problematic identifier, and unifying identifiers; if the extracted content includes a recognition error, useless space or line break, or a garbled code or omission, supplementing and modifying, by human beings, the extracted content to complete extraction and organization of the content of the relevant standard texts and form preliminary structured data.

Standard documents referenced in the present disclosure, whether dataset standards, subject code standards, or code value domain standards, all have different text structures. By referring to guidelines such as WS/T370-2022 Compilation Standard of Basic Health Information Dataset and T/CHIA6-2018 Specification for the Compilation of Specialized Electronic Medical Record Dataset, and considering differences between actual texts and national, industry, regional, and group standards, each type of text structure may undergo text analysis and content unit identification. Common content units across various types of standards are merged, and common features are extracted, while unique units are extracted separately. For different text structures, a database is designed for storing an extracted structured object.

Since a type of a text belongs to the unstructured text, which is mostly in .pdf or .doc formats, the recognition and extraction of text content may be carried out through the human-machine collaboration manner. A machine manner may mainly include the OCR image recognition and PDF content extraction techniques. For example, extracted text content includes a preface, introduction, prescribed content, scope of application, cited documents, terminology, acronyms, references, and so on. The extracted text content may be subjected to data cleaning, data review, and data quality control.

For example, for identifier data such as a standard number, an internal identifier, etc., a regular expression of the identifier data is written according to a clearly defined coding rule, spelling check and quality control on different codes are performed, a problematic identifier is corrected, and identifiers are unified, so as to facilitate normalization and statistics. The standard number is unique and may be used directly for the construction of the knowledge graph of data elements. However, identifiers of data elements may be duplicated in different standards, they cannot be used directly for identification, and need to be redefined as unique codes.

Additionally, extracted content may include recognition errors, unnecessary spaces, line breaks, garbled text, omissions, etc., which require manual supplementation and modification to complete the extraction and organization of entire text content, so as to form the preliminary structured data. The regular expression refers to a text pattern that describes a rule for string matching. The regular expression can facilitate retrieval, replacement, validation, and other operations on data elements.

In some embodiments, a specific process for extracting the entity type data and the attribute data from the unstructured text in the structured data includes:
manually annotating and performing review and quality control on the entity type by recognizing, extracting, and annotating from the unstructured text in the structured data using a field vocabulary or machine learning manner.

Not all entity types originate from the structured data, and data that characterize features of biomedical field standards may need to be extracted and annotated from unstructured descriptions (e.g., titles, abstracts, etc.) in the structured data using field vocabularies or machine learning manners. The entity types (e.g., diseases, departments, subject headings, etc.) need to be manually annotated and subjected to quality control, so as to enrich and enhance a field feature and an application scenarios feature of the data element standard and the data element, and then realize more fine-grained and multi-dimensional content revelation ranging from biomedical field standards to sets of data elements, data elements to value domains, and so on.

The concept identification of data elements: among dataset standards collected in the present disclosure, a small number of dataset standards (for example, a group standard issued by the Guangdong Provincial Hospital Association) cover specialized fields such as chronic diseases, hypertension, coronary heart disease, and cerebral infarction. The standard references a standard of ISO/IEC 11179 Metadata Registration System. For example, in a group standard such as T/GDPHA 031-2021 General Standard Dataset for Cerebrovascular Disease Research, mappings between data elements and concepts of data elements in vocabularies or common data element repositories such as CDISC, SNOMED CT, LOINC, and NIH CDE have been implemented, with concept English names or concept ID codes annotated. Therefore, a relationship between data elements and corresponding concepts of data elements may be extracted from such dataset standards.

The extraction of concepts of these data elements is based on English vocabularies/ontologies of medical field. However, most data elements in dataset standards do not define concepts of data elements and are expressed in Chinese. Therefore, the present disclosure uses Chinese/English vocabularies/ontologies of medical field to obtain the concepts of data elements. The subject vocabulary of medical field includes subject terms and entry terms, and has a hierarchical structure of concepts. Each subject term contains multiple entry terms with synonymous relationships. By matching data elements with subject terms and the entry terms under each subject term, the concept of the data element may be obtained.

In addition, if specific resources are involved, data must be extracted from texts or supplemented with external link information to ensure data association and resource accessibility. Specific resources may include reference papers, cited policies, cited standards, and other resources.

In some embodiments, the process for constructing types of semantic associative relationships between one or more entity types is as follows.

The following highlights a process for defining and processing relationships between a plurality of important entity types that need to be constructed.
(1) A relationship between data standards. The relationship between data standards is pluralistic. For example, a dataset standard references other standards, a new standard replaces a deprecated standard, a standard follows other standards, and so on. Additionally, a compositional relationship between standards is often overlooked. A biomedical dataset standard may include a plurality of standards. For example, an electronic medical record dataset for hypertension specialties includes 14 sections. Standards in these sections together form a dataset standard, and a relationship between these standards is that they are components of a same dataset. A value domain standard is similar. For example, WS 364 Health Information Data Element Value Domain Code includes 17 sections, such as demographic and socio-economic characteristics, health history, health risk factors, etc. Among the sections, except for the first and second sections, which are compilation rules, the remaining 15 sections are available code tables. The 15 sections together form a value domain of health information data elements. A relationship between data standards is shown in a table below:

TABLE 1
Relationship between data standards

| Entity type 1 | Relationship type | Entity type 2 |
|---|---|---|
| Dataset standard | Belong | Dataset standard |
| Dataset standards | Quote | Standard |
| Dataset standard (changes and | Replace | Dataset standard |
| modifications) | | |
| Dataset standard | Quote | Publications |
| Set of data elements | Replenish | Data elements |
| Set of data elements | Reuse | Data elements |
| Set of data elements | Include | Data elements |
| Dataset standard | Refer | Publications |
| Dataset standard | Include | Value domain |
| Dataset standard | Involve | Medical |
| | | scales/questionnaires |

(2) A relationship between a set of data elements and data elements. The set of data elements may be specifically reflected in a biomedical dataset standard as a set of specialized attributes for data elements with specific names. Each set of specialized attributes for data elements may typically include a plurality of data elements. The division of sets of data elements is ignored in existing research and applications. In the dataset standard, a specialized attribute of the data element includes the classification of the data element. For example, General Data Element Standards for Clinical Scientific Research on Gastric Cancer includes sets of seven specialized attributes of data elements. The sets of seven specialized attributes of data elements may include sets of: a general data element, demographic basic information of a subject, an outpatient (emergency) medical record of a subject, examination information of a subject, test information of a subject, admission and discharge information of a gastric subject, and adverse event information of a subject. Therefore, the data standard and the set of data elements may be in an inclusion relationship, and the set of data elements and the data elements may be in an inclusion relationship, the set of data elements may include a plurality of data elements.
(3) A relationship between data elements and concepts of data elements. Data elements may mainly originate from specific attributes of data elements, which most of the data elements in a standard of Chinese biomedical dataset do not provide concepts of data elements and information of subjects as required by a standard of ISO/IEC 11179 “Metadata Registration System”. This part needs to be supplemented with the concepts of data elements using a subject vocabulary of medical field, or the like.
(4) A relationship between data elements. The relationship between the data elements may include three types including a synonymous relationship, a relevant relationship, and an irrelevant relationship. Specifically, the relationship between the data elements is realized through following step 1) to step 6):
1) after identifying the concepts of the data elements, performing synonymous relationship recognition on the data elements, and if the concepts of two data elements in the data elements are the same in each subject vocabulary of a same medical field, determining the two data elements being in the synonymous relationship, and marking a similarity between the two data elements as 1;
2) if the two data elements are in a non-synonymous relationship, performing similarity calculation on the two data elements with completely different standard codes and data element identifiers using a Jaccard similarity manner by determining a ratio of an intersection set to a concatenation set between sets corresponding to the two data elements, respectively, wherein a calculation formula is as follows:

$$Sim\_ele ( E 1 , E 2 ) = \frac{\vert A ⋂ B \vert}{\vert A \vert + \vert B \vert - \vert A ⋂ B \vert} \qquad ( 1 )$$

where, E1 and E2 denote two data elements, respectively, a tokenization processing is performed on a text of each of the two data elements, E denotes a tokenized text composed of a data element name and a data element definition of the two data elements, Sim_ele_name( ) denotes a data element similarity, A denotes a tokenized text of E1, and B denotes a tokenized text of E2, and a final similarity result is controlled to be in a range of [0, 1]. The tokenized text refers to a separate lexical unit cut from a continuous text.
3) if the two data elements are in the non-synonymous relationship, calculating a similarity between a first data element and a second data element in the two data elements according to the calculation formula; if the similarity between the two data element s is greater than a data element synonymity threshold, determining the first data element and the second data elements being in a candidate synonymity relationship;
4) if the similarity between the two data elements is greater than a data element relevance threshold and less than the data element synonymity threshold, then determining the first data element and the second data element are being in a candidate relevance relationship;
5) if the similarity between the data elements is less than the data element relevance threshold, recording the similarity between the two data elements only, and marking a relationship between the first data element and the second data element as irrelevant;
6) a candidate relationship between each pair of data elements may not be obtained through similarity calculation alone, but also through manual verification and adjustment to determine an exact relationship to ensure the accuracy of the relationship. This establishes the multi-dimensional fine-grained correlation and degree of association between data elements, providing intelligent recommendations for subsequent creation and reuse of data elements.
(5) A relationship between data elements and value domains of the data elements. The value domain of the data element refers to a value range of the data element, e.g., a range of blood pressure. The present disclosure refines the relationship between the data element and the value domain of the data element, and divides a usage manner of the value domain of the data element at a fine-grained level. The value domain of the data element may be classified into four types including an enumeration with external reference type, an enumeration with internal reference type, an enumeration defined within a standard type, and a non-enumerated type based on a source of the value domain and the usage manner.

The enumeration with external reference type refers to referencing a value domain table of the other standard (different from the standard where the data element and the value domain are located), with a clear value domain standard or a table name provided.

The enumeration with internal reference type refers to referencing a value domain table defined within the same standard where data element and value domain are located, entries of allowed values being more than 4, with clearly specified table name and table code.

The enumeration defined within a standard type refers to that within a standard where the data element and the value domain are located, the allowed values are defined directly in the data element section without using a form of value domain table. Typically, a count of entries of the allowed values is fewer than 4.

The non-enumerated type refers to a value domain that is not listed by entries of the allowed values. The non-enumerated type may be typically identified using text to describe the allowed values or free-fill.

Based on the above definitions and methodology, a process for determining a relationship between the data elements and the value domains of the data elements may include Step k1 to Step k4:
Step k1, determining, based on the data element and a value domain corresponding to the data element, whether an allowed value of the data element includes a standard number or a number or name of a value domain code table, judging through a coding rule base, and in response to determining the allowed value including the standard number or the number or name of the value domain code table, determining the value domain of the data element as enumeration reference; or in response to determining the allowed value not including the standard number or the number or name of the value domain code table, performing following steps including:
Step k2, if the value domain of the data element is enumeration reference, further judging if a standard number of a dataset of the value domain is the standard number of the data element or if a number of a value domain code table of the value domain is the number of the value domain code table of the data element, and in response to determining the standard number of the dataset being different from the standard number of the data element or the number of the value domain code table of the value domain not including the number of the value domain code table of the data element, determining the value domain of the data element as the enumeration with external reference type; and in response to determining the standard number of the dataset being the same as the standard number of the data element or the number of the value domain code table of the value domain including the number of the value domain table of the data element, determining the value domain of the data element as the enumeration with internal reference type;
Step k3, in response to determining the allowed value of the data element not including the standard number or the number or name of the value domain code table and the allowed value includes “;” determining a split numeric item as the enumeration defined within a standard type; and
Step k4, in response to determining the split numeric item not belonging to the enumeration defined within a standard type, determining the split numeric item as the non-enumerated type.
(6) A relationship between a dataset standard and a medical scale/questionnaire. The medical scale may be used in the dataset standard, and a scale name and information of the medical scale may be extracted from a text, and a connection between a specific medical scale and data element is established by complementing resources of the medical scale.
(7) A relationship between a data element and a medical scale/questionnaire.

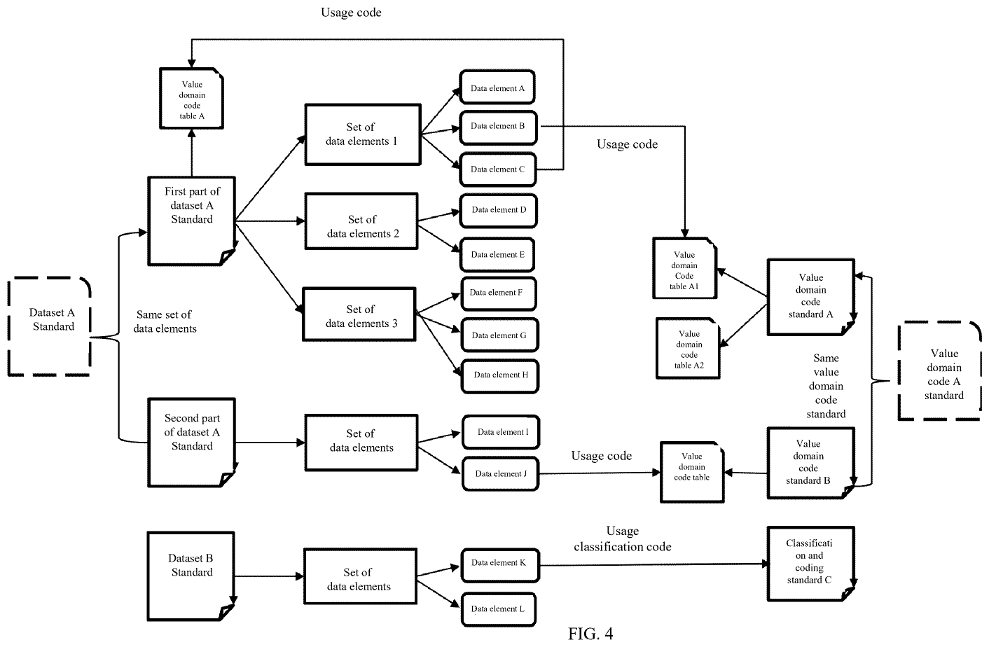

Specifically, a relationship between the dataset standard, the set of data elements, the data element, and the value domain code is shown in FIG. 4. FIG. 4 is a schematic diagram illustrating relationships between dataset standards, sets of data elements, data elements, and value domain codes according to some embodiments of the present disclosure.

In some embodiments, knowledge fusion of a plurality of types of data includes Step c1 to Step c4 as follows.
In some embodiments, a processor may achieve the knowledge fusion through knowledge merging, entity disambiguation, co-reference resolution, or the like. Example data of different entity types need to be de-duplicated and disambiguated, and targeted processing is performed based on the characteristics of different entity types.
Step c1, disambiguating using a pre-existing unique code, including processing a cross-level number. For example, in the data standard, even though a standard number is unique, different descriptions of a same standard (such as standard name, standard number, and abbreviation) may appear in different locations within a dataset standard document, which may lead to a same object not being recognized consistently.

Similarly, there are differences in a name of value domain code table, and a name of a vocabulary. For example, CV03.00.107, WS364.5CV03.00.107 Dietary Habits Code Table, and Patr 5 of WS364.5 Health Information Data Element Value Domain Code actually all correspond to a same value domain code table. There is also an issue of code duplication between internal and external codes, as there is currently no fine-grained Chinese data element query system available, which leads to the occurrence of code duplication.
Step c2, standardizing a name. Normalization and merging of a name is required because there are different expressions for a same organization name. For example, if Health Department Statistics Information Center, Ministry of Health Statistics Information Center of the People's Republic of China, and Health Ministry Health Statistics Information Center all refer to a same entity, then standardization and merging of the names are required. Therefore, the normalization of naming and coding is implemented through regulations and standards including WS/T306 Rules for Classification and Coding of Health Information Datasets and WS370-2012 Rules for Formulating Specifications for the Preparation of Basic Health Information Datasets, an institutional specification library and a field vocabulary, similarity calculation, manual verification, and quality control.

Terms, acronyms, etc., are also semantically merged through a field subject vocabulary, a general subject vocabulary, and so on. The field subject vocabulary refers to a systematic vocabulary list that is organized, classified, compiled, and arranged to cover all the subjects within a specific academic field. The general subject vocabulary may be a list of universally used field-specific terms.
Step c3, merging names of the data elements through similarity calculation between the data elements, merging of the concepts of the data elements, and manual discrimination.
Step c4, merging names of data value domain tables. The value domain table in a standard text of a dataset and an allowed value of the data element may be both related to a relevant name of the data value domain table including a table number, a table code, and a table name. It is required to perform structured processing on the table number, the table code, and the table name, correct, combine, and merge the table number, the table code, and the table name, and fuse a standard number to realize merging and disambiguation of the names of data value domain tables. The relevant name of the data value domain table may include a table number, a table code, and a table name. The standard number may be interpreted as a corresponding unique representer of an associated name of a data value domain table.

In some embodiments, the processor may also store and perform quality inspection on the knowledge graph.

In some embodiments, storage of the knowledge graph includes:
establishing a plurality of entity attribute tables and a plurality of entity triple relationship tables, performing batch conversion, importing, and converting triple data (e.g., subject, predicate, object) to UTF-8 to avoid encoding issues; and storing the knowledge graph with a Neo4j graph database. For importing data into the Neo4j graph database, a Neo4j-import tool may be used to import organized structured triple data to form a final knowledge graph. All data may then be queried and visualized through Cyber queries, supporting the query of a relationship between entity types in the knowledge graph of the standard data element of the biomedical dataset.

In some embodiments, data update includes the following content.

With the establishment of new standards for biomedical dataset and the revision of existing standards, content will change accordingly. Collection and processing of data related to dataset standards and data elements are ongoing, and updating and supplementing of example data corresponding to entity types for changed content are performed. The data elements, dataset standards, and institutions may be merged and a newly generated type of semantic associative relationship and data may be supplemented into the knowledge graph of the standard data element of the biomedical dataset.

Specifically, a specific embodiment is introduced to further explain the process.
(1) Designing an entity type and a relationship between entity types of a knowledge model, as shown in Table 2 and Table 3.

TABLE 2
Examples of entity types

| Serial | | |
|---|---|---|
| number | Entity type | Illustration |
| 1 | Data standard | A standard of for standardizing a dataset, value domain code, |
| | | classification code, etc. |
| 2 | Term | A term used in a standard |
| 3 | Abbreviation | An abbreviation used in a standard |
| 4 | Stipulated | Main technical content of standard content |
| | content | |
| 5 | Scope of | A scope of application, scenario, etc. of a standard |
| | application | |
| 6 | Preface | A corresponding preface section in a standard text |
| 7 | Introduction | A corresponding introductory section in a standard text, which |
| | | is not included in all standard texts |
| 8 | Domain | A secondary classification corresponding to a subject |
| | | classification |
| 9 | Publication | A reference of a standard text |
| 10 | Unit | A hospital department that may correspond to a certain disease |
| | | based on content of s standard |
| 11 | Disease | About a disease for which an ICD10 code is desirable, based on |
| | | content of a standard |
| 12 | Data element | A data element in a standard text |
| 13 | Concept of | A normalized name and source of a data element |
| | data element | |
| 14 | Responsible | A standardized name of a responsible institution |
| | institution | |
| 15 | Proposing | A standardized name of a proposing institution |
| | institution | |
| 16 | Drafting | A standardized name of a drafting institution |
| | institution | |
| 17 | Registering | A standardized Name of a registering institution |
| | institution | |
| 18 | Set of data | A categorized set of a plurality of data elements based on a |
| | elements | feature of data elements |
| 19 | Value domain | A range, source, etc. of a value of a data element |
| | code | |
| 20 | Drafter | A standardized name of a drafter |
| 21 | Medical scale | A medical scale is a standardized measurement tool consisting |
| | | of multiple items designed to reveal the level of theoretical |
| | | variables that are not amenable to direct methods of |
| | | measurement |

TABLE 3
Examples of relationship between entity types

| Serial | | | |
|---|---|---|---|
| number | Entity type 1 | Attributes of relationships | Entity type 2 |
| 1 | Standard | Quote | Standard |
| 2 | Standard | Belong | Standard |
| 3 | Standard | Adopt | Standard |
| 4 | Standard | Replace | Standard |
| 5 | Standard | Preface | Preface |
| 6 | Standard | Introduction | Introduction |
| 7 | Standard | Includes terminology | Terminology |
| 8 | Standard | Includes abbreviations | Abbreviation |
| 9 | Standard | Scope of application | Scope of application |
| 10 | Standard | Stipulated content | Stipulated content |
| 11 | Standard | Refer | Publications |
| 12 | Standard | Related units | Unit |
| 13 | Standard | Related Diseases | Disease |
| 14 | Standard | Relevant domains | Domain |
| 15 | Standard | Draft | Drafting institution |
| 16 | Standard | Publish | Publishing institution |
| 17 | Standard | Response | Responsible institution |
| 18 | Standard | Propose | Proposing institution |
| 19 | Standard | Draft | Drafter |
| 20 | Standard | Contains a set of data elements | Set of data elements |
| 21 | Set of data | Include | Data elements |
| | elements | | |
| 22 | Data element | Belong | Concept of the data |
| | | | element |
| 23 | Data element | Enumeration with external | Value domain code |
| | | reference | |
| 24 | Data element | Enumeration with internal | Value domain code |
| | | reference | |
| 25 | Data element | Enumeration defined within a | Value domain code |
| | | standard | |
| 26 | Data element | Non-enumerated | Value domain code |
| 27 | Data element | Reuse | Data element |
| 28 | Data element | Replenish | Data element |
| 29 | Standard | Include | Value domain code |

(2) An example of extracting structured entity types from structured data and unstructured data as shown in Table 4, Table 5 and Table 6.

TABLE 4
Extracted structured entity types and attributes-Standard example

| | |
|---|---|
| Standard number | WS 375.20-2016 |
| International | 11.020 |
| Standard | |
| Classification | |
| Number | |
| China Standard | C 07 |
| Classification | |
| Number | |
| Standard Chinese | [symbol] 20 [symbol] : [symbol] |
| name | |
| Standard type | Dataset standard |
| Standard English | Basic dataset of discase control-Part 20: Registration and report of |
| name | stroke |
| Catalog | Industry standard |
| Publisher | National Health and Family Planning Commission of the People's |
| | Republic of China |
| Preface | WS 375, Basic dataset of discase control, is now divided into the |
| | following parts: |
| | Part 1: Comprehensive AIDS Control; . . . |
| | This part is Part 20 of WS 375. This part is drafted in accordance with |
| | the rules given in GB/T 1.1-2009. |
| | Main drafting unit of this part: Shanghai Center for Disease Control and |
| | Prevention, Shanghai Institute of Preventive Medicine, Shanghai |
| | Municipal Health and Family Planning Commission, Shanghai |
| | Municipal Health and Family Planning Commission Information Center. |
| | Principal drafters of this part: Wu Fan, Miao Falcon, Li Xinjian, Zhong |
| | Weijian, Xia, Jiang Zhihai, Cai Chun, Xie Hua. |
| Drafting Institution | Shanghai Center for Disease Control and Prevention, Shanghai Institute |
| | of Preventive Medicine, Shanghai Municipal Health and Family |
| | Planning Commission, Information Center of Shanghai Municipal |
| | Health and Family Planning Commission |
| Stipulated content | WS 375 specifies attributes of metadata of datasets and attributes of data |
| | elements of a basic dataset of the registration and report of stroke. |
| Scope of | This part applies to disease prevention and control organizations, |
| application | medical institutions providing related services and health administrative |
| | departments for related business data collection, transmission and |
| | storage. |
| Disease | Stroke (ICD10/163.9) |
| Unit | neurology |
| Referenced | GB/T 2260 Administrative division codes of the People's Republic of |
| standard | China |
| | GB/T 2261.1 Classification and Coding of Personal Basic Information- |
| | Part 1; Sex Codes for Persons |
| | GB/T 2261.2 Classification and Codes of Basic Personal Information- |
| | Part 2: Marital Status Codes |
| | GB/T 3304 Roman Alphabet Spelling and Codes for the Names of |
| | China's Ethnic Groups |
| | GB/T 4658 Academic Calendar Codes |
| | GB/T 6565 Occupations Classification and Codes |
| | WS 364.3 Value domain codes of health information data elements-Part |
| | 3: Demographic and socio-economic characteristics |
| | WS 364.5 Value domain codes of health information data elements-Part |
| | 5: Health risk factors |
| | WS 364.11 Value domain codes of health information data elements- |
| | Part 11: Medical assessment |
| | WS 370 Specifications for the Preparation of Basic Health Information |
| | Datasets |
| | ICD-10 International Standard Classification of Diseases codes |

TABLE 5
Extracted structured entity types and attributes-Example of data elements

| | | | | Identi- | | |
|---|---|---|---|---|---|---|
| | | Chinese | | fication | Data | Allowable |
| | | name | | format | type | value of |
| External | Internal | of data | Definition of | of data | of data | data |
| identifier | identifier | element | data element | element | element | element |
| HDSD00. | DE05.01. | [symbol] | Codes for TCM | S3 | N1 | T/CIATCM |
| C6.003 | 501.00 | [symbol] | Clinical | | | 003-2019 |
| | | [symbol] | diagnostic | | | Chinese |
| | | | methods in | | | Medicine |
| | | | specific coding | | | Information |
| | | | systems | | | Data |
| | | | | | | Elements |
| | | | | | | Value |
| | | | | | | Domain |
| | | | | | | Code |
| | | | | | | 05.01.501 |
| | | | | | | Chinese |
| | | | | | | Medicine |
| | | | | | | Diagnosis |
| | | | | | | Code Table |
| HDSD00. | DE05.01. | [symbol] | Codes for TCM | S3 | N1 | T/CIATCM |
| C6.004 | 502.00 | [symbol] | clinicians use | | | 003-2019 |
| | | | vision to observe | | | Chinese |
| | | | abnormal | | | Medicine |
| | | | changes in the | | | Observation |
| | | | patient's spirit, | | | Code List |
| | | | color, shape, | | | of |
| | | | form, tongue, | | | Traditional |
| | | | excretions, and | | | Chinese |
| | | | pediatric | | | Medicine |
| | | | fingerprints in | | | Information |
| | | | order to | | | Data |
| | | | understand the | | | Elements |
| | | | diagnostic | | | Value |
| | | | content of the | | | Domain |
| | | | condition in | | | Code |
| | | | specific coding | | | |
| | | | systems | | | |

TABLE 6
Structured data elements and concepts of data elements

| | | | | | SNOMEDCT |
|---|---|---|---|---|---|
| | | Identifier of | Name of | CDISC concepts | concepts of |
| Standard | Internal | data | data | of data | data |
| number | identifier | element | element | elements | elements |
| T/GDPHA014- | LV010101.001. | DE02.01.00 | lymphatic | \ | C96660 |
| 2021 | 04.020 | 4.01 | node | | |
| | | | transfer | | |
| T/GDPHA013- | COPD010101.0 | DE02.01.00 | exertion | Forcevitalcapac- | C111361 |
| 2021 | 01.04.030 | 4.11 | spirometry | itymeasurement | |
| T/GDPHA013- | COPD010101. | DE02.01.00 | Exertion | FVCVolRespira- | |
| 2021 | 001.04.031 | 4.12 | spirometry | toryPredicted | |
| | | | predictions | | |

(3) Based on an association relationship between entity types constructed by the knowledge graph, generating a triple group, as shown in Table 7 and Table 8.

TABLE 7
Triple data of entity type

| SetID: | | |
|---|---|---|
| ID | Name | :LABEL |
| SetID1 | Specialized Attributes of Common Data Elements for Chronic | Set of data |
| | Disease Clinical Research Program Information | elements |
| SetID2 | Specialized Attributes of a subset of data elements for basic | Set of data |
| | demographic information of Subjects for Chronic Disease | elements |
| | Clinical Research | |
| SetID3 | Specialized Attributes of Data Elements of Outpatient | Set of |
| | (Emergency) Clinic Records of Subjects for Chronic Disease | data elements |
| | Clinical Research | |
| SetID4 | Specialized Attributes of Data Elements of Inspection | Set of data |
| | information of Subjects for Chronic Disease Clinical Research | elements |
| SetID5 | Specialized Attributes of Data Elements of Examination | Set of data |
| | information of Subjects for Chronic Disease Clinical Research | elements |

TABLE 8
Data of references of entity type standards

| StID: START_ID | StID: END_ID | :TYPE |
|---|---|---|
| T/GDPHA 002-2021 | GB/T 2261.1-2003 | Quote |
| T/GDPHA 002-2021 | GB/T 2261.2-2003 | Quote |
| T/GDPHA 002-2021 | GB/T 2261.4-2003 | Quote |
| T/GDPHA 002-2021 | GB/T 3304-1991 | Quote |

(4) Performing fusion and construction on a knowledge graph to achieve merging of entities through rules and dictionaries.

For example, CV03.00.107, WS364.5 CV03.00.107 Dietary Habits Code Table, WS364.5 Health Information Data Elements Value Domain Codes-Part 5, may be unified and merged into WS364.5 CV03.00.107 Dietary Habits Code Table.

As another example, the Statistical Information Center of the Ministry of Health, the Statistical Information Center of the Ministry of Health of the People's Republic of China, the Health Statistics Information Center of the Ministry of Health of the People's Republic of China may be merged into the Statistical Information Center of the Ministry of Health of the People's Republic of China.
(5) Storing data and performing quality inspection.

After importing all the triple data into the Neo4j graph database, a data sampling check is performed to verify the correctness of the triple data and to ensure that the entity types and association relationships are correct.

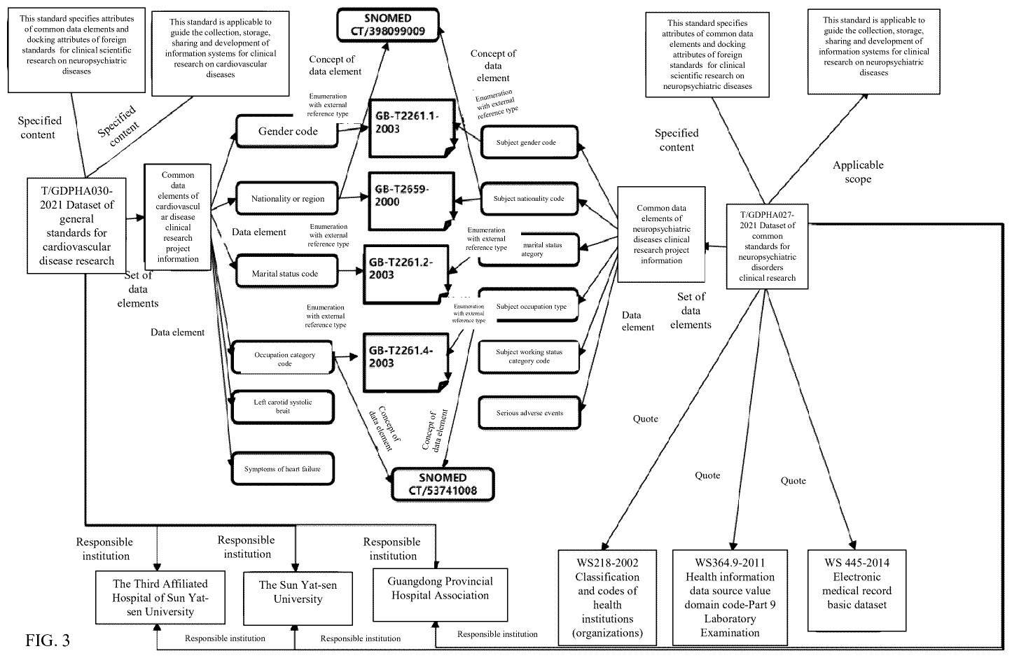

FIG. 3 is a schematic diagram illustrating example data of a portion of a knowledge graph according to some embodiments of the present disclosure.

Example data of a portion of a finally-constructed knowledge graph is shown in FIG. 3.

Embodiments of the present disclosure provide a system for constructing a knowledge graph of a standard data element of a biomedical dataset, including:
a data collection module, configured to collect relevant standard texts of data elements of different types of biomedical datasets and data of a relevant standard of the biomedical dataset;
a data analysis module, configured to analyze and summarize the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content;
a knowledge model construction module, configured to construct the knowledge model of the knowledge graph of the standard data element of the biomedical dataset, define one or more entity types, establish an attribute of each of the one or more entity types and types of semantic associative relationships between the one or more entity types;
an entity type extraction module, configured to extract entity type data and attribute data from structured data and an unstructured text in the structured data; and
a knowledge graph obtaining module, configured to obtain the knowledge graph of the standard data element of the biomedical dataset by performing knowledge fusion on a plurality of types of data based on the types of semantic associative relationships between the one or more entity types.

Each of the embodiments in the present disclosure is described in a progressive manner, and each embodiment focuses on the differences with other embodiments, and the same similar parts between each embodiment can be referred to each other. For the device disclosed in the embodiment, since it corresponds to the method disclosed in the embodiment, the description is relatively simple, and the relevant details can be referred to in the method section for further explanation.

The foregoing description of the disclosed embodiments enables a person skilled in the art to realize or use the present invention. Multiple modifications to these embodiments will be apparent to those skilled in the art, and the general principles defined herein may be realized in other embodiments without departing from the spirit or scope of the present invention. Accordingly, the present invention will not be limited to these embodiments shown herein, but will be subject to the broadest possible scope consistent with the principles and novel features disclosed herein. The basic concepts have been described above, and it will be apparent to those skilled in the art that the foregoing detailed disclosure is intended to be exemplary only, and does not constitute a limitation of the present disclosure. While not expressly stated herein, a person skilled in the art may make various modifications, improvements, and amendments to the present disclosure. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments of the present disclosure. Such as “an embodiment”, “one embodiment”, and/or “some embodiments” means a feature, structure, or characteristic associated with at least one embodiment of the present disclosure. Accordingly, it should be emphasized and noted that “an embodiment” or “one embodiment” or “an alternative embodiment” in different places in the present disclosure do not necessarily refer to the same embodiment. In addition, certain features, structures, or characteristics in one or more embodiments of the present disclosure may be suitably combined.

Furthermore, unless expressly stated in the claims, the order of the processing elements and sequences, the use of numerical letters, or the use of other names as described in the present disclosure are not intended to qualify the order of the processes and methods of the present disclosure. While the above disclosure discusses, by way of various examples, a number of embodiments of the present disclosure that are presently thought to be useful, it is to be understood that such detail serves only an illustrative purpose, and that the additional claims are not limited to the disclosed embodiments, rather, the claims are intended to cover all amendments and equivalent combinations that are consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be noted that in order to simplify the presentation of the present disclosure, and thereby aid in the understanding of one or more of the embodiments of the present disclosure, the foregoing descriptions sometimes combine a variety of features into a single embodiment, accompanying drawings, or description thereof. However, this method of disclosure does not imply that the objects of the present disclosure require more features than those mentioned in the claims. Rather, claimed subject matter may lie in less than all features of a single foregoing disclosed embodiment.

Some embodiments use numbers describing the number of components, attributes, and it should be understood that such numbers used in the description of embodiments are modified in some examples by the modifiers “approximately”, “nearly”, or “substantially”. Unless otherwise noted, the terms “approximately,” “nearly,” or “substantially” indicates that a ±20% variation in the stated number is allowed. Correspondingly, in some embodiments, the numerical parameters used in the present disclosure and claims are approximations, which approximations are subject to change depending on the desired characteristics of the individual embodiment. In some embodiments, the numerical parameters should consider the specified number of valid digits and utilize a general digit retention method. While the numerical domains and parameters used to confirm the breadth of their ranges in some embodiments of the present disclosure are approximations, in specific embodiments, such values are set to be as precise as practicable.

For each of the patents, patent applications, patent application disclosures, and other materials cited in the present disclosure, such as articles, books, specification sheets, publications, documents, etc., the entire contents of which are hereby incorporated herein by reference. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions and/or use of terms in the present disclosure shall prevail.

Finally, it should be understood that the embodiments described in the present disclosure are only used to illustrate the principles of the embodiments of the present disclosure. Other deformations may also fall within the scope of the present disclosure. As such, alternative configurations of embodiments of the present disclosure may be viewed as consistent with the teachings of the present disclosure as an example, not as a limitation. Correspondingly, the embodiments of the present disclosure are not limited.

1. A method for constructing a knowledge graph of a standard data element of a biomedical dataset, comprising:
collecting relevant standard texts of data elements of different types of biomedical datasets and data of a relevant standard of the biomedical dataset;
analyzing and summarizing the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content;
wherein the analyzing and summarizing the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content includes: analyzing and summarizing the relevant standard texts of the data elements of the different types of biomedical datasets and the data of the relevant standard of the biomedical dataset by performing optical character recognition (OCR) on the relevant standard texts of the data elements of the different types of biomedical datasets and parsing the relevant standard texts using a natural language processing (NLP) manner to obtain structured data and unstructured text;
constructing the knowledge model of the knowledge graph of the standard data element of the biomedical dataset, defining one or more entity types, establishing an attribute of each of the one or more entity types and types of semantic associative relationships between the one or more entity types, including:
extracting entity type data and attribute data from structured data and an unstructured text;
wherein a process for extracting the entity type data and the attribute data from the structured data includes:
recognizing and extracting content of the relevant standard texts of the data elements using a human-machine collaboration manner; performing data cleaning, data review, and data quality control on the extracted content, writing a regular expression of identifier data according to a clearly defined coding rule, performing spelling check and quality control on different codes, correcting a problematic identifier, and unifying identifiers; if the extracted content includes a recognition error, useless space or line break, or a garbled code or omission, supplementing and modifying, by human beings, the extracted content to complete extraction and organization of the content of the relevant standard texts and form preliminary structured data;
obtaining the knowledge graph of the standard data element of the biomedical dataset by performing knowledge fusion on a plurality of types of data based on the types of semantic associative relationships between the one or more entity types; wherein
the types of semantic associative relationships between the one or more entity types include: a relationship between data standards, a relationship between a set of data elements and the data elements, a relationship between the data elements and concepts of the data elements, a relationship between the date elements, a relationship between the data elements and value domains of the data elements, a relationship between a dataset standard and a medical scale/questionnaire, and a relationship between the data elements and the medical scale/questionnaire; wherein the relationship between data standards is pluralistic; the data standards and the dataset of data elements are in an inclusion relationship, and the dataset of data elements and the data elements are in an inclusion relationship, the dataset of data elements includes a plurality of data elements; and the relationship between the data elements includes a synonymous relationship, a relevant relationship, and an irrelevant relationship; the value domains of the data elements are classified into four types including an enumeration with external reference type, an enumeration with internal reference type, an enumeration defined within a standard type, and a non-enumerated type based on a source of the value domains and a usage manner; the medical scale is used in the dataset standard, and a scale name and information are extracted from a text, and a connection between a specific medical scale and the data element is established by complementing resources of the medical scale; each of the data elements is a storage name in a standardized dataset of the medical scale, and an association between the data elements and the specific medical scale is established;
a process for determining the relationship between the data elements includes:
after identifying the concepts of the data elements, performing synonymous relationship recognition on the data elements, and if the concepts of two data elements in the data elements are the same in each subject vocabulary of a same medical field, determining the two data elements being in the synonymous relationship, and marking a similarity between the two data elements as 1;
if the two data elements are in a non-synonymous relationship, performing similarity calculation on the two data elements with completely different standard codes and data element identifiers using a Jaccard similarity manner by determining a ratio of an intersection set to a concatenation set between sets corresponding to the two data elements, respectively, wherein a calculation formula is as follows:
$$Sim\_ele ( E 1 , E 2 ) = \frac{\vert A ⋂ B \vert}{\vert A \vert + \vert B \vert - \vert A ⋂ B \vert}$$
where, E1 and E2 denote the two data elements, respectively, a tokenization processing is performed on a text of each of the two data elements, E denotes a tokenized text composed of a data element name and a data element definition of each of the two data elements, Sim_ele_name( ) denotes a data element similarity, A denotes a tokenized text of E1, and B denotes a tokenized text of E2, and a final similarity result is controlled to be in a range of [0, 1];
if the two data elements are in the non-synonymous relationship, calculating a similarity between a first data element and a second data element in the two data elements according to the calculation formula; if the similarity between the two data elements is greater than a data element synonymity threshold, determining the first data element and the second data elements being in a candidate synonymity relationship;
if the similarity between the two data elements is greater than a data element relevance threshold and less than the data element synonymity threshold, determining the first data element and the second data element being in a candidate relevance relationship;
if the similarity between the data elements is less than the data element relevance threshold, recording the similarity between the two data elements, and marking a relationship between the first data element and the second data element as irrelevant;
a process for determining the relationship between the data elements and the value domains of the data elements and determining the types of the value domains includes:
for each of the data elements,
determining, based on the data element and a value domain corresponding to the data element, whether an allowed value of the data element includes a standard number or a number or name of a value domain code table, judging through a coding rule base, and in response to determining the allowed value including the standard number or the number or name of the value domain code table, determining the value domain of the data element as enumeration reference; or in response to determining the allowed value not including the standard number or the number or name of the value domain code table, performing following steps including:
if the value domain of the data element is enumeration reference, further judging if a standard number of a dataset of the value domain is the standard number of the data element or if a number of a value domain code table of the value domain is the number of the value domain code table of the data element, and in response to determining the standard number of the dataset being different from the standard number of the data element or the number of the value domain code table of the value domain not including the number of the value domain code table of the data element, determining the value domain of the data element as the enumeration with external reference type; and in response to determining the standard number of the dataset being the same as the standard number of the data element or the number of the value domain code table of the value domain including the number of the value domain table of the data element, determining the value domain of the data element as the enumeration with internal reference type;
in response to determining the allowed value of the data element not including the standard number or the number or name of the value domain code table and the allowed value includes “;” determining a split numeric item as the enumeration defined within a standard type; and
in response to determining the split numeric item not belonging to the enumeration defined within a standard type, determining the split numeric item as the non-enumerated type;
the method further comprising storing the knowledge graph, the storing includes establishing a plurality of entity attribute tables and a plurality of entity triple relationship tables, performing batch conversion, importing and converting triple data to Unicode Transformation Format-8-bit to avoid encoding issues, and storing the knowledge graph using a graph database; wherein the graph database supports querying converted data and visualizing query results to users, and the triple relationship tables represent semantic relationships between different entities.

2. The method of claim 1, further comprising:
obtaining the structured data and the unstructured text by performing optical character recognition (OCR) on the relevant standard texts of the data elements of the different types of the biomedical datasets and parsing the relevant standard texts using a natural language processing (NLP) manner.

3. The method of claim 1, further comprising storing and performing quality inspection on the knowledge graph, wherein
the quality inspection includes after importing the triple data into the graph database, performing data sampling to verify correctness of the triple data to ensure correctness of an entity type and a relevance relationship.

4. (canceled)

5. The method of claim 1, wherein a process for extracting the entity type data and the attribute data from the unstructured text includes:
manually annotating and performing review and quality control on the entity type by recognizing, extracting, and annotating from the unstructured text using a field vocabulary or machine learning manner.

6. The method of claim 1, wherein the performing the knowledge fusion on the plurality of types of data includes:
disambiguating using a pre-existing unique code, including processing a cross-level number;
standardizing a name, including normalization of naming and coding through regulations and standards including WS/T306 Rules for Classification and Coding of Health Information Datasets, WS370-2012 Rules for Formulating Specifications for the Preparation of Basic Health Information Datasets, an institutional specification library and a field vocabulary, similarity calculation, manual verification, and quality control; semantically merging a term and acronym through a field subject vocabulary and a general subject vocabulary; wherein WSIT306 Rules for Classification and Coding of Health Information Datasets, WS370-2012 Rules for Formulating Specifications for the Preparation of Basic Health Information Datasets is China health industry standards;
merging names of the data elements through similarity calculation between the data elements, merging of the concepts of the data elements, and manual discrimination; and
merging names of data value domain tables, wherein a value domain table in a standard text of a dataset and the allowed value of the data element are both related to a relevant name of the data value domain table including a table number, a table code, and a table name, and the merging names of data value domain tables including: performing structured processing on the table number, the table code, and the table name, correcting, combining, and merging the table number, the table code, and the table name, and fusing a standard number to realize merging and disambiguation of the names of the data value domain tables.

7. A system for constructing a knowledge graph of a standard data element of a biomedical dataset, wherein the system is applied to the method of claim 1, and the system comprises:
a data collection circuit, configured to collect the relevant standard texts of the data elements of the different types of the biomedical datasets and the data of the relevant standard of the biomedical dataset;
a data analysis circuit, configured to analyze and summarize the relevant standard texts of the data elements of the different types of biomedical datasets and the data of the relevant standard of the biomedical dataset to construct the knowledge model of the knowledge graph of the standard data element of the biomedical dataset, parse the data, and extract the fine-grained content;
wherein the analyzing and summarizing the relevant standard texts of the data elements of the different types of biomedical datasets and the data of the relevant standard of the biomedical dataset to construct a knowledge model of the knowledge graph of the standard data element of the biomedical dataset, parse the data, and extract fine-grained content includes: analyzing and summarizing the relevant standard texts of the data elements of the different types of biomedical datasets and the data of the relevant standard of the biomedical dataset by performing optical character recognition (OCR) on the relevant standard texts of the data elements of the different types of biomedical datasets and parsing the relevant standard texts using a natural language processing (NLP) manner to obtain structured data and unstructured text;
a knowledge model construction circuit, configured to construct the knowledge model of the knowledge graph of the standard data element of the biomedical dataset, define the one or more entity types, establish the attribute of each of the one or more entity types and the types of semantic associative relationships between the one or more entity types;
an entity type extraction circuit, configured to extract the entity type data and the attribute data from the structured data and the unstructured text;
wherein extracting the entity type data and the attribute data from the structured data includes:
recognizing and extracting content of the relevant standard texts of the data elements using a human-machine collaboration manner; performing data cleaning, data review, and data quality control on the extracted content, writing a regular expression of identifier data according to a clearly defined coding rule, performing spelling check and quality control on different codes, correcting a problematic identifier, and unifying identifiers: if the extracted content includes a recognition error, useless space or line break, or a garbled code or omission, supplementing and modifying, by human beings, the extracted content to complete extraction and organization of the content of the relevant standard texts and form Preliminary structured data:
a knowledge graph obtaining circuit, configured to obtain the knowledge graph of the standard data element of the biomedical dataset by performing the knowledge fusion on the plurality of types of data based on the types of semantic associative relationships between the one or more entity type;
wherein the type of semantic associative relationships between the one or more entity types include: the relationship between the data standards, the relationship between the set of data elements and the data elements, the relationship between the data elements and the concepts of the data elements, the relationship between the date elements, the relationship between the data elements and the value domains of the data elements, the relationship between the dataset standard and the medical scale/questionnaire, and the relationship between the data elements and the medical scale/questionnaire; wherein the relationship between data standards is pluralistic; the data standards and the dataset of data elements are in the inclusion relationship, and the dataset of data elements and the data elements are in the inclusion relationship, the dataset of data elements includes the plurality of data elements; and the relationship between the data elements includes the synonymous relationship, the relevant relationship, and the irrelevant relationship; the value domains of the data elements are classified into four types including the enumeration with external reference type, the enumeration with internal reference type, the enumeration defined within a standard type, and the non-enumerated type based on the source of the value domains and the usage manner; the medical scale is used in the dataset standard, and the scale name and information are extracted from the text, and a connection between the specific medical scale and the data element is established by complementing resources of the medical scale; each of the data elements is the storage name in the standardized dataset of the medical scale, and an association between the data elements and the specific medical scale is established;
to determine the relationship between the data elements, the knowledge graph obtaining circuit is further configured to:
after identifying the concepts of the data elements, perform the synonymous relationship recognition on the data elements, and if the concepts of the two data elements are the same in each subject vocabulary of a same medical field, determine the two data elements in the synonymous relationship, and mark the similarity between the two data elements as 1;
if the two data elements are in the non-synonymous relationship, preform the similarity calculation on the two data elements with completely different standard codes and data element identifiers use the Jaccard similarity manner by determine the ratio of the intersection set to the concatenation set between the sets corresponding to the two data elements, respectively, wherein a calculation formula is as follows:
$$Sim\_ele ( E 1 , E 2 ) = \frac{\vert A ⋂ B \vert}{\vert A \vert + \vert B \vert - \vert A ⋂ B \vert}$$
where, E1 and E2 denote the two data elements, respectively, the tokenization processing is performed on the text of each of the two data elements, E denotes the tokenized text composed of the data element name and the data element definition of a data element, Sim_ele_name( ) denotes the data element similarity, A denotes the tokenized text of E1, and B denotes the tokenized text of E2, and the final similarity result is controlled to be in a range of [0, 1];
if the two data elements are in the non-synonymous relationship, calculate the similarity between the first data element and the second data element between the two data elements according to the calculation formula; if the similarity between the two data elements is greater than the data element synonymity threshold, determine the first data element and the second data elements are in the candidate synonymity relationship;
if the similarity between the two data elements is greater than the data element relevance threshold and less than the data element synonymity threshold, determine the first data element and the second data element in the candidate relevance relationship;
if the similarity between the data elements is less than the data element relevance threshold, recording the similarity between the two data elements, and mark the relationship between the first data element and the second data element as irrelevant;
to determine the relationship between the data elements and the value domains of the data elements and determining the types of the value domains, the knowledge graph obtaining circuit is further configured to:
determine, based on the data element and the value domain corresponding to the data element, whether the allowed value of the data element includes the standard number or the number or name of the value domain code table, judge through the coding rule base, and in response to determine the allowed value including the standard number or the number or name of the value domain code table, determine the value domain of the data element as enumeration reference; or in response to determine the allowed value not including the standard number or the number or name of the value domain code table, perform following steps include;
if the value domain of the data element is enumeration reference, further judge if the standard number of the dataset of the value domain is the standard number of the data element or if the number of the value domain code table of the value domain includes the number of the value domain code table of the data element, and in response to determine the standard number of the dataset is different from the standard number of the data element or the number of the value domain code table of the value domain not include the number of the value domain code table of the data element, determine the value domain of the data element as the enumeration with external reference type; and in response to determine the standard number of the dataset is the same as the standard number of the data element or the number of the value domain code table of the value domain include the number of the value domain table of the data element, determine the value domain of the data element as the enumeration with internal reference type;
in response to determine the allowed value of the data element not include the standard number or the number or name of the value domain code table and the allowed value includes “;” determine a split numeric item as the enumeration defined within a standard type; and
in response to determine the split numeric item not belong to the enumeration defined within a standard type, determine the split numeric item as the non-enumerated type;
the knowledge graph obtaining circuit further configured to: store the knowledge graph, the store includes establish a plurality of entity attribute tables and a plurality of entity triple relationship tables, perform batch conversion, import and convert triple data to Unicode Transformation Format-8-bit to avoid encode issues, and store the knowledge graph use a graph database; wherein the graph database supports query converted data and visualize query results to users, and the triple relationship tables represent semantic relationships between different entities.
